(19) United States
(12) Patent Application Publication
(10) Pub. No.: US 2014/0045202 A1
(43) Pub. Date: Feb. 13, 2014

(54) Analyte Sensors and Methods for Making and Using the Same

(71) Applicant: Abbott Diabetes Care Inc., Alameda, CA (US)

(72) Inventor: Yi Wang, San Ramon, CA (US)

(21) Appl. No.: 13/949,991

(22) Filed: Jul. 24, 2013

Related U.S. Application Data
(60) Provisional application No. 61/680,816, filed on Aug. 8, 2012.

(51) Int. Cl. G01N 33/66

(52) U.S. Cl. CPC G01N 33/66
USPC 435/14; 435/287.1

(57) ABSTRACT

The present disclosure provides an analyte sensor that includes a conductive calibration pattern which provides species-specific calibration information to a meter. Also provided, are analyte sensors comprising a layer of absorbent material disposed on a first major surface of a first substrate and/or a second major surface of a second substrate. Methods for making and using the analyte sensors are also provided.

### CROSS-REFERENCE TO RELATED APPLICATIONS

This application claims priority based on U.S. Provisional Application No. 61/680,816, filed Aug. 8, 2012, the disclosure of which is incorporated by reference herein in its entirety.

### INTRODUCTION

Analyte measurement is routinely used to monitor the level of analytes in a subject using an analyte sensor and an analyte meter. The process of using an analyte measurement device is not complicated, and is often performed several times a day. First, a user inserts an analyte test strip into a strip port of the measurement device. The user then places a sample to be analyzed into the analyte test strip. The measurement device analyzes the sample. The measurement device then typically displays the analyte level from the analysis.

A measurement device may need to be calibrated to a specific analyte sensor inserted or to calculate an analyte concentration based on the type of sample applied to the analyte sensor. A user-friendly method for calibrating a meter is of interest.

In order to ensure an accurate measurement is being generated, it is necessary to keep the measurement device free from contamination. There are instances where the strip port may become contaminated with blood or other fluids (e.g., calibration fluid). When this occurs, the performance of the measurement device suffers and the user is no longer assured an accurate result. As such, the user may need to purchase a new measurement device.

Dedicated hospital meters have high occurrence rates of contamination due to factors such as heavy use, need for calibration, and other environmental factors. Contamination of a hospital meter, and the subsequent need to replace the hospital meter, is costly.

The analyte sensors, measurement devices, and systems described herein address these needs as well as others.

### SUMMARY

The present disclosure provides an analyte sensor that includes a conductive calibration pattern which provides species-specific calibration information to a meter.

Also provided herein, are analyte sensors comprising a layer of absorbent material disposed on a first major surface of a first substrate and/or a second major surface of a second substrate.

Methods for making and using the analyte sensors are also provided.

In some embodiments, the analyte sensor may comprise a first substrate comprising a first major surface and a second major surface and a second substrate comprising a first major surface and a second major surface such that the second major surface of the first substrate faces the first major surface of the second substrate to provide a sample chamber, the sample chamber comprising a first electrode disposed on the second major surface of the first substrate or the first major surface of the second substrate and a second electrode disposed on the second major surface of the first substrate or the first major surface of the second substrate, wherein the sample chamber is positioned at a proximal end of the analyte sensor and wherein a distal end of the analyte sensor is connectable to a meter; a conductive calibration pattern on the first major surface of the first substrate and/or second major surface of second substrate at or near the distal end, wherein the calibration pattern provides a species-specific calibration information to the meter, wherein the conductive calibration pattern is one of a plurality of patterns, wherein the plurality of patterns comprise different patterns which provide different species-specific calibration information to a meter.

In some embodiments, the sensor further comprises a graphic disposed on the first major surface of the first substrate and/or second major surface of second substrate, wherein the graphic indicates the species compatible for the analyte sensor. In certain cases, the species-specific calibration information is for a non-human species. In some cases, the species-specific calibration information is for a feline, canine, equine, or murine species.

In some embodiments, the shape of the calibration pattern may provide the species-specific calibration information. In other cases, resistance of the calibration pattern provides the species-specific calibration information.

In other cases, calibration information comprises prompting the meter to require input of a calibration code.

A system for analysis of an analyte in a sample is also provided. The system may comprise an analyte sensor comprising a first substrate comprising a first major surface and a second major surface and a second substrate comprising a first major surface and a second major surface such that the second major surface of the first substrate faces the first major surface of the second substrate to provide a sample chamber, the sample chamber comprising a first electrode disposed on the second major surface of the first substrate or the first major surface of the second substrate and a second electrode disposed on the second major surface of the first substrate or the first major surface of the second substrate, wherein the sample chamber is positioned at a proximal end of the analyte sensor and wherein a distal end of the analyte sensor is connectable to a meter; a conductive calibration pattern on the first major surface of the first substrate and/or second major surface of second substrate at or near the distal end, wherein the calibration pattern provides a species-specific calibration information to the meter; wherein the conductive calibration pattern is one of a plurality of patterns, wherein the plurality of patterns comprise different patterns which provide different calibration information to a meter; a meter comprising a port comprising contacts for contacting the calibration pattern.

A method of providing species-specific calibration code on an analyte sensor is also provided. The method comprises performing sensor calibration on at least one of a batch of analyte sensors to determine a species-specific calibration code for the analyte sensor; disposing a conductive calibration pattern on a surface of analyte sensors in the batch, wherein the calibration pattern provides the species-specific calibration code to a meter, wherein the conductive calibration pattern is one of a plurality of calibration patterns, wherein the plurality of patterns comprise different patterns which provide different calibration information to a meter.

Also provided herein is an analyte sensor comprising a layer of absorbent material; a first substrate comprising a first major surface and a second major surface; a second substrate comprising a first major surface and a second major surface; a sample chamber present between the second major surface of the first substrate and the first major surface of the second substrate, wherein the layer of absorbent material is disposed on the first major surface of the first substrate and/or the second major surface of the second substrate. In certain embodiments, the absorbent layer may comprise a disinfectant. In some cases, the absorbent layer comprises a mesh layer.

A method of using an analyte sensor is also disclosed. The method comprises inserting the analyte sensor into a sensor port of a meter; and contacting a sample with the analyte sensor, wherein the analyte sensor comprises a layer of absorbent material; a first substrate comprising a first major surface and a second major surface; a second substrate comprising a first major surface and a second major surface; a sample chamber present between the second major surface of the first substrate and the first major surface of the second substrate, wherein the layer of absorbent material is disposed on the first major surface of the first substrate and/or the second major surface of the second substrate, wherein the layer of absorbent material absorbs any excess sample that may enter the sensor port of the meter and/or any liquid present in the sensor port of the meter.

In certain cases, the absorbent layer may comprise a disinfectant. The method may further comprises contacting a site on skin of a subject with the absorbent layer and lancing the site to provide a sample before contacting the sample with the analyte sensor.

A system for analyte analysis is disclosed. The system comprises a meter; and an absorbent strip, wherein the absorbent strip is configured to substantially fill a sensor port of the meter. In certain cases, the absorbent strip comprises an indicator that provides an indication that a liquid has been absorbed by the absorbent strip.

A method of manufacturing an analyte sensor is also described. In some cases, the method comprises, bringing together a first substrate comprising a first major surface and a second major surface and a second substrate comprising a first major surface and a second major surface such that the second major surface of the first substrate faces the first major surface of the second substrate to provide a sample chamber, the sample chamber comprising a first electrode disposed on the second major surface of the first substrate or the first major surface of the second substrate and a second electrode disposed on the second major surface of the first substrate or the first major surface of the second substrate, wherein the sample chamber is positioned at a proximal end of the analyte sensor and wherein a distal end of the analyte sensor is connectable to a meter; determining calibration code for the analyte senor by performing sensor calibration; disposing a non-conductive graphic design on the first major surface of the first substrate and/or second major surface of second substrate, wherein the graphic design indicates to a user the type of analyte sensor; disposing a conductive calibration pattern on the first major surface of the first substrate and/or second major surface of second substrate, wherein the calibration pattern provides the calibration code to the meter. In some cases, disposing the non-conductive graphic design and the conductive insertion monitor comprises digital printing. In other cases, disposing the conductive insertion monitor comprises screen printing.

### BRIEF DESCRIPTION OF THE DRAWINGS

A detailed description of various embodiments of the present disclosure is provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol that is used in one drawing to refer to a particular element may be used in another drawing to refer to a like element.

FIGS. 1A, 1B, and 1C illustrate analyte sensors with different calibration patterns.

FIG. 2 depicts an analyte sensor with an absorbent layer.

FIG. 3 illustrates an analyte sensor with an absorbent layer inserted in a port of the analyte meter.

FIG. 4A shows an absorbent strip. FIG. 4B shows individual layers of absorbent strip.

### DETAILED DESCRIPTION OF THE INVENTION

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be embodied by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The present disclosure is controlling to the extent there is a contradiction between the present disclosure and a publication incorporated by reference.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

Analyte Sensors with Custom Calibration Pattern and Methods of Making the Same

A method of manufacturing an analyte sensor with custom calibration pattern is provided. The method comprises providing a plurality of sensors, each of the sensor comprising a first substrate comprising a first major surface and a second major surface and a second substrate comprising a first major surface and a second major surface such that the second major surface of the first substrate faces the first major surface of the second substrate to provide a sample chamber, the sample chamber comprising a first electrode disposed on the second major surface of the first substrate or the first major surface of the second substrate and a second electrode disposed on the second major surface of the first substrate or the first major surface of the second substrate, wherein the sample chamber is positioned at a proximal end of the analyte sensor and wherein a distal end of the analyte sensor is connectable to a meter; determining calibration code for the analyte senor by performing sensor calibration; disposing a conductive calibration pattern on the first major surface of the first substrate and/or second major surface of second substrate at the distal end of the sensor, wherein the calibration pattern encodes calibration information, wherein the conductive calibration pattern is one of a plurality of conductive calibration patterns, wherein each of the plurality of conductive calibration patterns encode different calibration information.

In general, the electrodes as well as the turn-on bar are disposed on the substrates for manufacturing analyte sensors at the beginning of the manufacturing process. The turn-on bar functions to connect to contacts of a measuring device and turn it on. The measuring device (e.g., meter) has a fixed calibration information which determines the method for calculating the concentration of an analyte in a sample applied to the sensor. The analyte sensor is configured such that it will conform to the calibration information present in the meter. This configuring of the sensor to conform to the calibration information present in the meter is usually done by performing calibration of one of the sensors from a batch of sensors and physically modifying the other sensors in the batch such that they all have a standard calibration which corresponds to the calibration information in the meter used with the sensor. This procedure is described in U.S. Pat. No. 7,866,026, which is incorporated by reference herein.

However, in many instances, it is not possible to adjust the calibration of the sensors so that they are compatible with a meter with a fixed calibration code. Such sensors have to be discarded. In addition, the step of physically modifying the sensors complicates the manufacturing process.

In another aspect, the turn-on bar disposed on a sensor substrate at the beginning of the manufacturing process becomes scratched or otherwise compromised during the manufacturing resulting in defective sensors.

The method of manufacturing described herein overcomes the above described problems and provides additional benefits.

In the method of manufacturing analyte sensors described herein, the conductive calibration pattern is disposed towards the end of the manufacturing process after sensor calibration has been performed. Disposing conductive calibration pattern towards the end of the manufacturing process provides the dual benefit of avoiding compromising the pattern during the manufacturing process and disposing a calibration pattern that corresponds to the calibration of the sensor, which calibration pattern is encoded to provide calibration information to the meter. A different calibration pattern conveys a different calibration information to the meter.

In certain embodiments, the method further comprises disposing a non-conductive graphic design on the first major surface of the first substrate and/or second major surface of second substrate, wherein the graphic design indicates to a user the type of analyte sensor.

In certain cases, disposing the non-conductive graphic design and/or the conductive calibration pattern comprises digitally printing the non-conductive graphic design and/or the conductive calibration pattern.

In certain embodiments, disposing the calibration pattern comprises screen printing.

In certain embodiments, the first and second substrates can be a sheet or a continuous roll on a web. The multiple working electrodes can be formed the first substrate and multiple counter electrodes can be formed the second substrate. The first and second substrates brought together and the individual sensors may then be separated out. In some embodiments, a plurality of working and counter electrodes may be disposed on the first, the second substrate providing a cover, the two substrates brought together and the individual sensors may then be separated out. In yet other embodiments, the working and counter electrodes may be disposed on the same substrate, the substrate can be scored and folded to bring the working electrodes and counter electrodes in a facing orientation. Details of these steps are provided in U.S. 2002/0053523, published May 9, 2002, incorporated by reference herein.

In certain embodiments, a spacer layer may separate the working and counter electrodes. In certain embodiments, a spacer layer may separate co-planar electrodes from the cover layer.

An adhesive spacer is formed over at least one of the substrate/working electrode and substrate/counter electrode(s). The adhesive spacer may be a single layer of adhesive or a double-sided adhesive tape (e.g., a polymer carrier film with adhesive disposed on opposing surfaces). To form the channel, the spacer, optionally provided with one or more release liners, may be cut (e.g., die-cut) to remove the portion of the adhesive corresponding to the channel prior to disposing the spacer on the substrate. Alternatively, the adhesive may be printed or otherwise disposed on the substrate according to a pattern which defines the channel region. The thickness of the spacer typically determines the spacing between the working and counter electrodes. When the uniformity of this spacing among sensors is necessary (e.g., for coulometric measurements), uniformity in the thickness of the spacer is important. Preferably, the thickness does not vary more than +/−5% over the individual sensor and/or among individual sensors in a batch.

The non-leachable or diffusible redox mediator and/or second electron transfer agent are disposed onto the substrate in the sample chamber region. The redox mediator and/or second electrode transfer agent can be disposed independently or together on the substrate prior to or after disposition of the spacer. The redox mediator and/or second electrode transfer agent may be disposed by a variety of methods including, for example, screen printing, ink jet printing, spraying, painting, striping along a row or column of aligned and/or adjacent electrodes, and the like. Other components may be deposited separately or with the redox mediator and/or second electrode transfer agent including, for example, surfactants, polymers, polymer films, preservatives, binders, buffers, and cross-linkers.

After disposing the spacer, redox mediator, and second electron transfer agent, the substrate can be folded to form the sensor. The faces of the substrate are joined by the adhesive of the spacer. After bringing the faces together, the sensor can be cut out using a variety of methods including, for example, die cutting, slitting, or otherwise cutting away the excess substrate material and separating the individual sensors. In some embodiments, a combination of methods may be used. For example, some features may be die cut, while the remainder of the sensor is cut by slitting. As another alternative, the sensor components may first be cut out of the substrates and then brought together to form the sensor by adhesively joining the two components using the spacer adhesive.

In certain cases, one or more of the sensors may be used in an assay to determine calibration of the sensors of a batch of sensors. A calibration code usually includes a slope and y-intercept values. The slope and y-intercept values are used to determine the analyte concentration based on the measured signal. The calibration code is needed to standardize the analysis results received from non-standardized sensors. In other words, different sensors vary, e.g., from lot to lot, a sufficient amount that, if no compensation were made, the results would differ from sensor to sensor and the results could be clinically inaccurate. Details of methods for determining calibration of sensor are provided in U.S. 2009/0229122, published Sep. 17, 2009, which is incorporated herein by reference.

Upon determining the calibration code of the sensor, a conductive calibration pattern that corresponds to the calibration code is disposed on the remaining sensors of the batch of sensors.

The individual sensors may then be produced by die-cutting, for example.

In general, a plurality of predetermined patterns are available, where each pattern corresponds to a particular calibration code and one of the predetermined patterns are disposed on the distal end of the sensor.

Also provided herein is a system for analysis of an analyte in a sample, the system comprising an analyte sensor comprising a conductive calibration pattern; a meter comprising a first contact, a second contact, and a third contact for connection to the calibration pattern, wherein the conductive calibration pattern is configured to complete a circuit between i) the first and second contact to provide a first calibration code to the meter, or ii) the second and third contact to provide a second calibration code to the meter, or iii) the first and second contact, and the first and the third contact, and the second and third contact to provide a third calibration code to the meter.

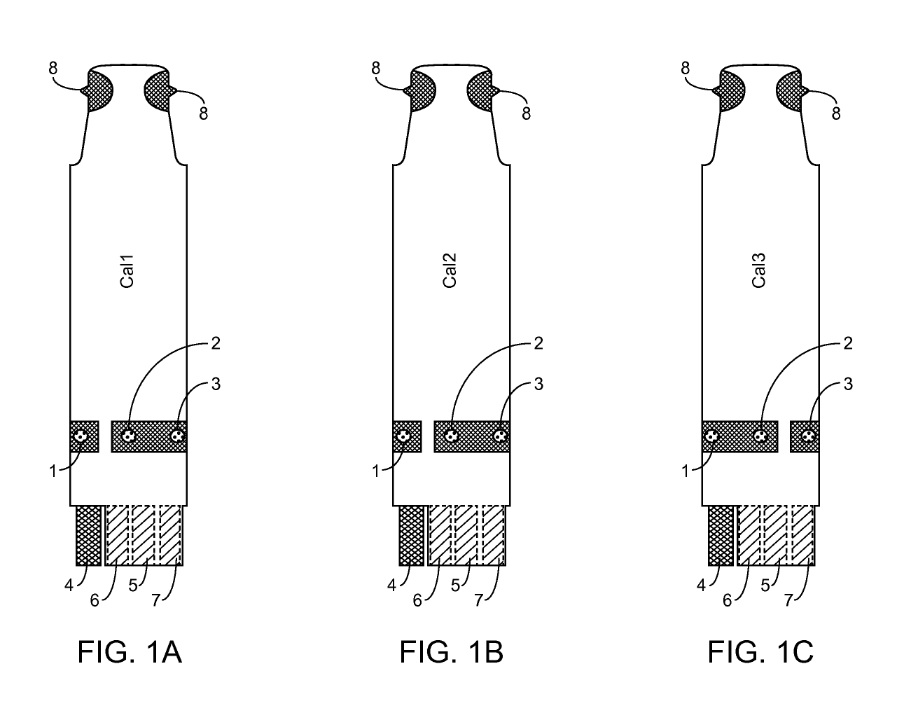

Exemplary conductive calibration patterns are illustrated in FIGS. 1A, 1B and 1C. FIGS. 1A, 1B and 1C illustrate analyte sensors cal 1, cal 2, and cal 3, respectively. These sensors require different calibration codes encoded by the different conductive calibration patterns. A measuring device for calculating the concentration of an analyte present in a sample in the sensor may include three contacts where each of the circuits are formed between two or three of the different contacts. For example, in the sensors of FIG. 1A, 1B and 1C, first, second, and third contacts of a meter (not shown) contact at the area 1, area 2, and area 3 (indicated by circles). In sensor cal 1, a circuit is completed only between the second and third contacts of the meter, conveying to the meter that the sensor corresponds to calibration code 1. In sensor cal 2, a circuit is completed between the first and second contact, and the first and the third contact, and the second and third contact, conveying to the meter that the sensor corresponds to calibration code 2. In sensor cal 3, a circuit is completed only between the first and second contacts of the meter, conveying to the meter that the sensor corresponds to calibration code 3. In the example in FIGS. 1A, 1B and 1C, a single meter is compatible with all three different calibration coded sensors.

Although, the shape of the analyte sensor as well as the configuration of the electrodes of the analyte sensor may vary, the analyte sensors depicted in FIGS. 1A, 1B, and 1C, include a working electrode with a working electrode tab 4 for connection to a fourth contact in the meter, a counter electrode with a counter electrode tab 5 for connection to a fifth contact in the meter and indicator electrodes with contact tables 6 and 7 for connection to a sixth and seven contact of the meter. The working electrode in analyte sensors illustrated in FIGS. 1A, 1B, and 1C is in facing configuration to the counter and indicator electrodes. The analyte sensors of FIGS. 1A, 1B, and 1C are side fill sensors in which a sample that may be filled in the sample chamber by using a protrusion 8.

The analyte sensors with different calibration codes may be compatible with different species, samples from which may be analyzed using the system described above. For example, the notation of cal code 1, cal code 2, and cal code 3 on the analyte sensor or on a container containing the analyte sensor may indicate to a user that the analyte sensor is compatible with a first species, a second species, and a third species, respectively. In other embodiments, the analyte sensor instead of or in addition to being associated (by an indication on the sensor and/or indication on a container holding the sensor) with a notation, such as, cal code 1, may be associated with a marking or indication that may be an image representative of the species or a one, two, or three letter code for the species in a language understood by the user. For example, the marking associated with the analyte sensor may indicate to a user the animal species compatible with the sensor. In certain cases, the marking may be a graphic associated with the analyte sensor, such as an image representative of the species or a one, two, or three letter code for the species in a language understood by the user.

### Analyte Sensor and System for Clean Sensor Port

In another aspect, an analyte sensor that facilitates a clean sensor port in a measuring device for the analyte sensor is provided. The analyte sensor comprises a layer of absorbent material; a first substrate comprising a first major surface and a second major surface; a second substrate comprising a first major surface and a second major surface; a sample chamber present between the second major surface of the first substrate and the first major surface of the second substrate, wherein the layer of absorbent material is disposed on the first major surface of the first substrate and/or the second major surface of the second substrate.

In certain embodiments, the absorbent layer may include a disinfectant.

In further aspects of the analyte sensor, the absorbent layer may comprise a mesh layer.

In a further aspect, a method of using an analyte sensor is provided. The method comprises inserting the analyte sensor into a sensor port of a meter; and contacting a sample with the analyte sensor, wherein the analyte sensor comprises a layer of absorbent material; a first substrate comprising a first major surface and a second major surface; a second substrate comprising a first major surface and a second major surface; a sample chamber present between the second major surface of the first substrate and the first major surface of the second substrate, wherein the layer of absorbent material is disposed on the first major surface of the first substrate and/or the second major surface of the second substrate, wherein the layer of absorbent material absorbs any excess sample that may enter the sensor port of the meter and/or any liquid present in the sensor port of the meter.

In a further embodiment, the method comprises contacting a site on skin of a subject with the absorbent layer and lancing the site to provide a sample before contacting the sample with the analyte sensor.

Also provided herein is a system for analyte analysis, the system comprising a meter; and an absorbent strip, wherein the absorbent strip is configured to substantially fill a sensor port of the meter.

In certain cases, the absorbent strip comprises an indicator that provides an indication that a liquid has been absorbed by the absorbent strip.

In certain embodiments, the absorbent strip comprises a first layer of mesh, a second layer of absorbent material and a third layer of a substrate.

In certain embodiments, the system further comprises an analyte sensor that is operably connectable to the meter when inserted into the sensor port.

The absorbent layer of a sensor or of an absorbent strip may comprise any absorbent material, such as, cotton, nylon, cellulose, polymer gels, absorbent resin, or a combination thereof.

The absorbent material may be attached to a first substrate of a sensor or a second substrate of the sensor. In certain cases, the absorbent strip may be constructed of a first layer of a non-absorbent substrate, such as, a plastic substrate, e.g., a melinex substrate. A layer of absorbent material may be provided on the substrate. The layer of absorbent material may be attached to the substrate via an adhesive. In certain cases, a layer of a mesh may be disposed on the absorbent material layer. In certain cases, a layer of absorbent material may be disposed on both surfaces of the substrate, and further a layer of mesh may be disposed on both layers of absorbent material. In other cases, a layer of absorbent material may be disposed on both surfaces and the edges of the substrate, and further the whole absorbent material may be encased in a mesh layer.

In certain embodiments, the absorbent strip may be adhered to a first or a second substrate of a sensor. In other cases, a layer of absorbent material may be provided on may be provided on the first or second substrate of the sensor. The layer of absorbent material may then be covered with a mesh layer.

In certain embodiments, the absorbent strip may have the same dimensions as an analyte sensor. Accordingly, the absorbent strip may be inserted into the sensor port of a measurement device prior to and/or after the analyte sensor is inserted into the measurement device.

In some embodiments, the absorbent layer of an analyte sensor may be disposed at a location on the analyte sensor, which does not enter into the sensor port of the measurement device, such as, a meter. Rather, the absorbent layer covers the entry to the sensor port once the analyte sensor has been inserted into the port.

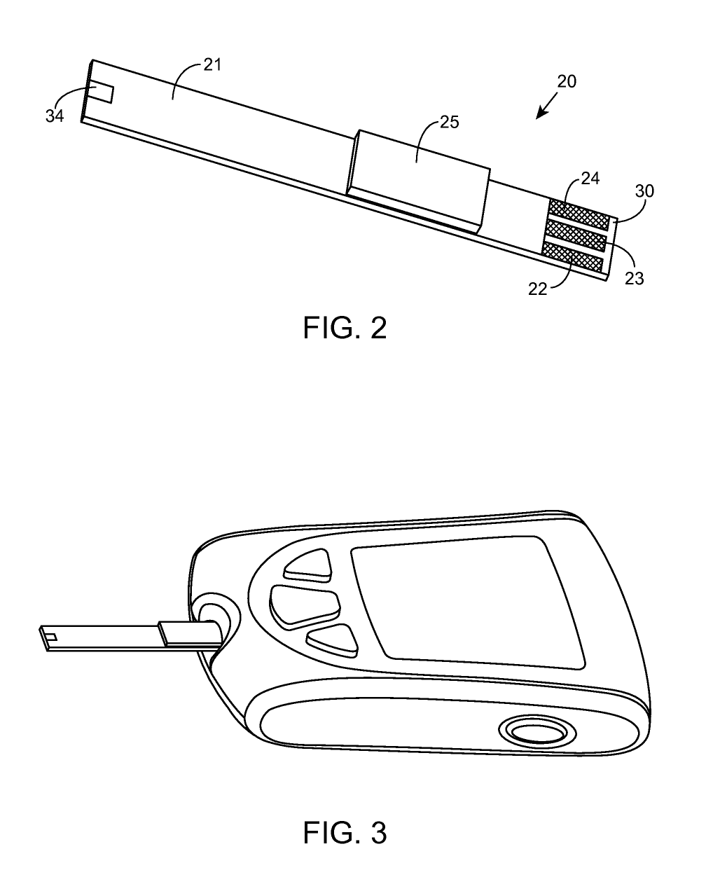

An exemplary embodiment of an analyte sensor comprising an absorbent layer is depicted in FIG. 2. FIG. 2 illustrates an analyte sensor 20 comprising a cover layer 21 disposed over co-planar electrodes 22, 23, and 24, which are present on substrate 30. The sample chamber may be filled by applying a sample to sample inlet 34. Although, the sensor in FIG. 2 includes certain configuration of the sample chamber and electrodes, it is understood that the sensor including the absorbent layer may have another configuration, such as, facing electrodes, side-fill sample chamber, and the like. An absorbent layer 25 is attached to the cover layer.

An illustration of a meter into which the analyte sensor has been inserted is provided in FIG. 3. As is evident from the figure, the absorbent layer blocks the sensor port such that any sample applied to the analyte sensor does not enter the sensor port. Although not depicted in FIG. 2 or 3, a layer of absorbent material, with a layer of a mesh disposed thereupon may be present on the other side of the sensor to provide an analyte sensor with absorbance capability on both sides. For example, a layer of absorbent material may be disposed on the cover 21.

In certain embodiments, the analyte sensor may also include a calibration code on a major surface of one or more substrates as described above.

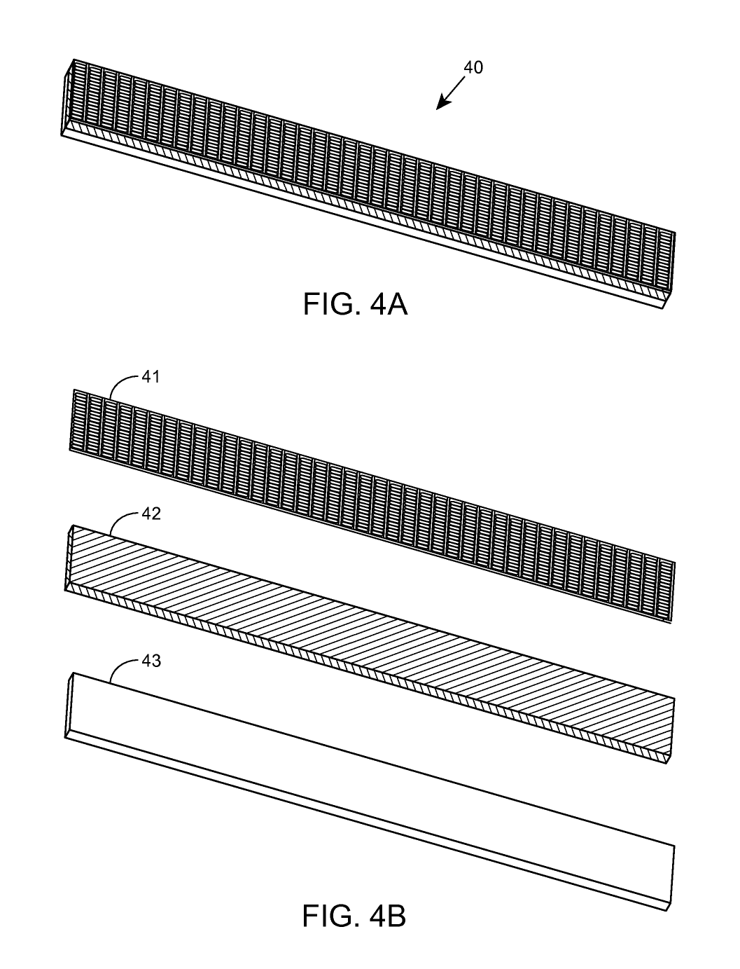

An exemplary embodiment of an absorbent strip comprising a substrate, layer of absorbent material, and a layer of a mesh is depicted in FIGS. 4A and 4B. FIG. 4A shows the assembled absorbent strip 40, while FIG. 4B shows the individual layers of the absorbent strip 40: a mesh layer 41, an absorbent material 42, and a substrate layer 43. Although not depicted in the figure, a layer of absorbent material, with a layer of a mesh disposed thereupon may be present on the other side of the substrate to provide an absorbent strip with absorbance capability on both sides.

Analyte Sensor with Optically Readable Code

In another aspect of the invention, an analyte sensor with an optically readable code made from non-conductive material is provided. The analyte sensor comprises a first substrate comprising a first major surface and a second major surface; a second substrate comprising a first major surface and a second major surface; a sample chamber present between the second major surface of the first substrate and the first major surface of the second substrate; the sample chamber comprising a first electrode disposed on the second major surface of the first substrate or the first major surface of the second substrate and a second electrode disposed on the second major surface of the first substrate or the first major surface of the second substrate; wherein the sample chamber is positioned at a proximal end of the analyte sensor and wherein a distal end of the analyte sensor is connectable to a meter; an optically readable code, wherein the optically readable code is present on the first major surface of the first substrate and/or on the second major surface of the second substrate at or near the distal end and provides information regarding the analyte sensor to a user or a meter, wherein the optically readable code is non-conductive and is not contacted by electrical connectors of the meter.

In certain cases, the optically readable code is readable by the meter and indicates to the meter the identity of the analyte sensor and/or calibration information for the analyte sensor.

In other cases, the optically readable code is readable by the user and indicates to the user the identity of the analyte sensor and/or calibration information for the analyte sensor.

In certain cases, the optically readable code is a geometric area printed with a defined density of a color, wherein the density indicates the identity and/or calibration information to the meter.

In certain cases, the optically readable code is a geometric area printed with a defined density of a color, wherein the color indicates the identity and/or calibration information to the meter.

In certain cases, the identity of the analyte sensor comprises information about type, manufacturing date, or lot identity of the analyte sensor.

In certain aspects, the analyte sensor further comprises a conductive turn-on bar disposed on the distal end of the analyte sensor.

Also provided herein is a method for performing analysis of an analyte in a sample, the method comprising contacting the sample with an analyte sensor comprising a first substrate comprising a first major surface and a second major surface; a second substrate comprising a first major surface and a second major surface; a sample chamber present between the second major surface of the first substrate and the first major surface of the second substrate; the sample chamber comprising a first electrode disposed on the second major surface of the first substrate or the first major surface of the second substrate and a second electrode disposed on the second major surface of the first substrate or the first major surface of the second substrate; wherein the sample chamber is positioned at a proximal end of the analyte sensor and wherein a distal end of the analyte sensor is connectable to a meter; an optically readable code, wherein the optically readable code is present on the first major surface of the first substrate and/or on the second major surface of the second substrate at or near the distal end and provides information regarding the analyte sensor to a user or a meter, wherein the optically readable code is non-conductive and is not contacted by electrical connectors of the meter; reading the optically readable code to obtain information regarding the analyte sensor; using the information to analyze the analyte present in the sample.

In certain aspects, the reading comprises reading by a meter.

In certain aspects, the reading comprises reading by a user.

In other embodiments, the using comprises applying a calibration code to calculation of concentration of analyte.

In a further embodiment, the using comprises identifying the analyte sensor and selecting a calculation algorithm for the analyte sensor.

In a further example, the applying is performed by a user or a meter.

In another example, the identifying and selecting is done by a meter or a user.

### Analyte Sensors

Generally, embodiments of the present disclosure relate to analyte sensors as described above and including electrodes present in co-planar or facing configuration.

In general, an analyte sensor strip has a first, proximal end and an opposite, distal end. At proximal end, sample to be analyzed is applied to the sensor. Proximal end could be referred as “the fill end” or “sample receiving end”. Distal end of the sensor is configured for operable connection to a device such as a meter. Sensor strip may be a layered construction, in certain embodiments having a generally rectangular shape, which is formed by first and second substrates.

Sensor strips include a sample chamber having an inlet or sample chamber entrance for access to sample chamber. Sensor strip may be a a tip-fill sensor, having inlet at proximal end.

Sample chamber may be defined by first and second substrates and a spacer. Generally opposite to inlet, through one or both of the substrates is a vent to allow egress of air from sample chamber upon displacement by a sample filling the sample chamber.

A sensor includes at least one working electrode with sensing chemistry material(s) thereon and a counter electrode. The counter electrode may also function as a reference electrode or the sensor may include a separate reference electrode. Optionally, a mesh layer may also be present in the sample chamber.

A sensing layer may be disposed in the sample chamber at a location adjacent to the electrodes or on one or more of the electrodes in the sample chamber. The sensing layer may include an analyte specific enzyme and a redox mediator. The sensing layer may be disposed as a thin (˜5 μm) film. In one embodiment, the sensing layer may span the working, counter, and reference electrodes.

The spacer layer may be a thin (˜100 μm) polymer tape layer with pressure-sensitive adhesive (PSA) on both surfaces. The spacer layer defines the dimensions (height and surface area) and shape of the sample chamber and the area of the sensing layer that is exposed to the biological sample.

A biological sample application/receiving area may in one embodiment be a notch cut in the end of the electrode strip to identify to the user the point at which the biological sample should be applied. This receiving area may also define the entrance to the sample chamber and promote ingress of the sample into the chamber.

In certain embodiments, the sample application/receiving area may be a protrusion that extends from the substrate that may define the entrance to the sample chamber and promote ingress of the sample into the chamber. The protrusion may be formed by a substrate and a cover to provide a tab which may be used to contact a sample for filling the sample chamber.

The sensor strips may also include an optional insertion monitor. The insertion monitor functions to wake-up or turn-on a meter once the sensor is operably connected to the meter. The insertion monitor may also provide calibration information to the meter, as described above.

Substrates/cover layer of the analyte sensors may be made of a polymer, such as a polyester (e.g., Mylar™ and polyethylene terephthalate (PET)), polyethylene, polycarbonate, polypropylene, nylon, polyvinyl chloride (PVC), polyurethanes, polyethers, polyamides, polyimides, or copolymers of these thermoplastics, such as PETG (glycol-modified polyethylene terephthalate).

In some embodiments, the sensors are made using a relatively rigid substrate, for example, to provide structural support against bending or breaking. Examples of rigid materials that may be used as the substrate include glass, poorly conducting ceramics, such as aluminum oxide and silicon dioxide.

The electrodes may be made of any conductive material such as pure metals or alloys, or other conductive materials. Examples include aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (such as highly doped polycrystalline silicon), silver, tantalum, tin, titanium, tungsten, uranium, vanadium, zinc, zirconium, mixtures thereof, and alloys or metallic compounds of these elements. In certain embodiments, the conductive material includes carbon, gold, platinum, palladium, iridium, or alloys of these metals, since such noble metals and their alloys are unreactive in biological systems.

Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like.

In certain embodiments, the thickness of spacer layer may be constant throughout, and may be at least about 0.01 mm (10 μm) and no greater than about 1 mm or about 0.5 mm. For example, the thickness may be between about 0.02 mm (20 μm) and about 0.2 mm (200 μm). In one certain embodiment, the thickness is about 0.05 mm (50 μm), and about 0.1 mm (100 μm) in another embodiment.

The sample chamber has a volume sufficient to receive a sample of biological fluid therein. In some embodiments, the sample chamber has a volume that is typically no more than about 1 μL, for example no more than about 0.5 μL, and also for example, no more than about 0.3 μL, 0.25 μL, or 0.1 μL.

The sensing layer may include deposited as an aqueous solution of an analyte specific enzyme and a redox mediator. The sensing layer can be screen-printed, deposited using an ink-jet printer, for example. The sensing layer may be disposed in the sample chamber on the working electrode and/or the reference and/or the counter electrode.

A layer of mesh may overlay the electrodes. This layer of mesh may protect the printed components from physical damage. The layer of mesh may also facilitate wetting the electrodes by reducing the surface tension of the sample, thereby allowing it to spread evenly over the electrodes. The mesh layer may be made of a polymer.

Analyte test strips for use with the invention can be of any kind, size, or shape known to those skilled in the art; for example, FREESTYLE® and FREESTYLE LITE™ test strips, as well as PRECISION™ test strips sold by ABBOTT DIABETES CARE Inc. In addition to the embodiments specifically disclosed herein, the reagents and methods of the present disclosure can be configured to work with a wide variety of analyte test strips, e.g., those disclosed in U.S. patent application Ser. No. 11/461,725, filed Aug. 1, 2006; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; U.S. Patent Application No. 12/102,374, filed Apr. 14, 2008, and U.S. Patent Application Publication No. 2009/0095625; U.S. Pat. No. 6,616,819; U.S. Pat. No. 6,143,164; U.S. Pat. No. 6,592,745; U.S. Pat. No. 6,071,391 and U.S. Pat. No. 6,893,545; U.S. Patent Application Publication No. U.S. 2007/0272563; U.S. Pat. No. 5,628,890; U.S. Pat. No. 6,764,581; and U.S. Pat. No. 7,311,812, for example, the disclosures of each of which are incorporated by reference herein in their entirety.

Analyte sensors are disclosed in these patent application publication and patents are each herein incorporated by reference in its entirety.

The terms “working electrode”, “counter electrode”, “reference electrode” and “counter/reference electrode” are used herein to refer to a portion or portions of a conductive trace which are configured to function as a working electrode, counter electrode, reference electrode or a counter/reference electrode respectively. In other words, a working electrode is that portion of a conductive trace which functions as a working electrode as described herein, e.g., that portion of a conductive trace which is exposed to an environment containing the analyte or analytes to be measured and not covered by an insulative layer (such as a spacer layer, a tape, or a cover), and which, in some cases, has been modified with one or more sensing layers as described herein. Similarly, a reference electrode is that portion of a conductive trace which function as a reference electrode as described herein, e.g., that portion of a conductive trace which is exposed to an environment containing the analyte or analytes to be measured and not covered by an insulative layer, and which, in some cases, includes a secondary conductive layer, e.g., a Ag/AgCl layer. A counter electrode is that portion of a conductive trace which is configured to function as a counter electrode as described herein, e.g., that portion of a conductive trace which is exposed to an environment containing the analyte or analytes to be measured and not covered by an insulative layer. As noted above, in some embodiments, a portion of a conductive trace may function as either or both of a counter electrode and a reference electrode.

The dimensions of the analyte sensor may vary. In certain embodiments, the overall length of analyte sensor may be no less than about 10 mm and no greater than about 50 mm. For example, the length may be between about 30 and 45 mm; e.g., about 30 to 40 mm. It is understood, however that shorter and longer sensor strips could be made. In certain embodiments, the overall width of sensor strip may be no less than about 3 mm and no greater than about 15 mm. For example, the width may be between about 4 and 10 mm, about 5 to 8 mm, or about 5 to 6 mm. In one particular example, sensor strip has a length of about 32 mm and a width of about 6 mm. In another particular example, sensor strip has a length of about 40 mm and a width of about 5 mm. In yet another particular example, sensor strip has a length of about 34 mm and a width of about 5 mm.

Representative examples of analyte specific enzymes that may be present in the sample chamber of the analyte sensors in a sensing layer include glucose dehydrogenase, glucose-6-phosphate dehydrogenase, glucose oxidase, cholesterol oxidase, lactate oxidase β-hydroxybutyrate dehydrogenase, alcohol dehydrogenase, lactate dehydrogenase, formaldehyde dehydrogenase, malate dehydrogenase, and 3-hydroxysteroid dehydrogenase. For example, an enzyme, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

Representative examples of redox mediators that may be present in the sample chamber of the analyte sensor, for example, in a sensing layer, include organometallic redox species such as metallocenes including ferrocene or inorganic redox species such as hexacyanoferrate (III), ruthenium hexamine, etc. Additional suitable electron transfer agents usable a redox mediators in the sensors of the present invention are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same.

Additional examples include those described in U.S. Pat. Nos. 6,736,957, 7,501,053 and 7,754,093, the disclosures of each of which are incorporated herein by reference in their entirety.

### Application of the Analyte Sensor

Also provided herein is a method of determining concentration of an analyte in a sample supplied to the sample chamber of the analyte sensors described herein.

In one embodiment, the determining step may include determining the concentration of the analyte by amperometry, coulometry, potentiometry, and/or voltametry, including square wave voltammetry.

A common use for an analyte sensor of the present invention is for the determination of analyte concentration in a biological fluid, such as blood, interstitial fluid, and the like, in a patient or other user. Analytes that may be determined include but are not limited to, for example, glucose, acetyl choline, amylase, bilirubin, cholesterol, chorionic gonadotropin, glycosylated hemoglobin (HbA1c), creatine kinase (e.g., CK-MB), creatine, DNA, fructosamine, glucose, glutamine, growth hormones, hormones, ketones, ketone bodies, lactate, peroxide, prostate-specific antigen, prothrombin, RNA, thyroid stimulating hormone, and troponin. In certain cases, the analyte sensor determines the concentration of glucose.

The analyte sensors as disclosed herein may be available at pharmacies, hospitals, clinics, from doctors, and other sources of medical devices. Multiple analyte sensors as disclosed herein may be packaged together and sold as a single unit; e.g., a package of about 25, about 50, or about 100 sensors, or any other suitable number. A kit may include one or more sensors, and additional components such as control solutions and/or lancing device and/or meter, etc.

The analyte sensors may be used for an electrochemical assay, or, for a photometric test. The analyte sensor may be used to provide the concentration of an analyte present in a body fluid sample by using a coulometric technique, a potentiometric technique or an amperometric technique. In certain embodiments, the sensor is connected to an amperometer to detect and provide concentration of an analyte, e.g., glucose. Sensors are generally configured for use with an electrical meter, which may be connectable to various electronics. As mentioned above, the meter may be a coulometer, a potentiometer or an amperometer. A meter may be available at generally the same locations as the sensors, and sometimes may be packaged together with the sensors, e.g., as a kit.

Examples of suitable electronics connectable to the meter include a data processing terminal, such as a personal computer (PC), a portable computer such as a laptop or a handheld device (e.g., personal digital assistants (PDAs)), and the like. The electronics are configured for data communication with the receiver via a wired or a wireless connection. Additionally, the electronics may further be connected to a data network (not shown) for storing, retrieving and updating data corresponding to the detected analyte level (e.g., glucose level) of the user.

The various devices connected to the meter may wirelessly communicate with a server device, e.g., using a common standard such as 802.11 or Bluetooth RF protocol, or an IrDA infrared protocol. The server device could be another portable device, such as a Personal Digital Assistant (PDA) or notebook computer, or a larger device such as a desktop computer, appliance, etc. In some embodiments, the server device has a display, such as a liquid crystal display (LCD), as well as an input device, such as buttons, a keyboard, mouse or touch-screen. With such an arrangement, the user can control the meter indirectly by interacting with the user interface(s) of the server device, which in turn interacts with the meter across a wireless link.

The server device may also communicate with another device, such as for sending data from the meter and/or the service device to a data storage or computer. For example, the service device could send and/or receive instructions (e.g., an insulin pump protocol) from a health care provider computer. Examples of such communications include a PDA synching data with a personal computer (PC), a mobile phone communicating over a cellular network with a computer at the other end, or a household appliance communicating with a computer system at a physician's office.

A lancing device or other mechanism to obtain a sample of biological fluid, e.g., blood, from the patient or user may also be available at generally the same locations as the sensors and the meter, and sometimes may be packaged together with the sensor and/or meter, e.g., as a kit.

The sensors are particularly suited for inclusion in an integrated device, i.e., a device which has the sensor and a second element, such as a meter or a lancing device, in the device. The integrated device may be based on providing an electrochemical assay or a photometric assay. In some embodiments, sensors may be integrated with both a meter and a lancing device. Having multiple elements together in one device reduces the number of devices needed to obtain an analyte level and facilitates the sampling process. For example, embodiments may include a housing that includes one or more of the sensor strips, a skin piercing element and a processor for determining the concentration of an analyte in a sample applied to the strip. A plurality of sensors may be retained in a cassette in the housing interior and, upon actuation by a user, a single sensor may be dispensed from the cassette so that at least a portion extends out of the housing for use.

### Operation of the Analyte Sensor

In use, a sample of biological fluid is provided into the sample chamber of the sensor, where the level of analyte is determined. The analysis may be based on providing an electrochemical assay or a photometric assay. In many embodiments, it is the level of glucose in blood that is determined. Also in many embodiments, the source of the biological fluid is a drop of blood drawn from a patient, e.g., after piercing the patient's skin with a lancing device, which could be present in an integrated device, together with the sensor strip.

Prior to providing the sample to the sensor, or even after providing the sample to the sensor, there may be no need for the user to input a calibration code or other information regarding the operation and/or interaction of the sensor with the meter or other equipment. The sensor may be configured so that the results received from the analysis are clinically accurate, without the user having to adjust the sensor or the meter. The sensor is physically configured to provide accurate results that are repeatable by a batch of sensors.

In certain cases, the user may determine the type of analyte sensor by looking at a graphic disposed on the sensor before applying a sample to the sensor. For example, the user may determine that the sample to be analyzed is from a first species, the user may then use an analyte sensor that has an indication that informs the user that the sensor is for use with a sample from the first species. For example, the first species may be a feline species, the analyte sensor for use with samples from feline species may be identified by an indication, such as, a marking on the sensor or a container holding the sensor. In certain cases, the marking or indication may be an image representative of the species or a one, two, or three letter code for the species in a language understood by the user. For example, the analyte sensor or container holding the sensor may have an image of a cat or the marking C which may indicate to a user that that the sensor is to be used for analyzing cat samples.

As explained above, a single meter may be used to analyze different types of analyte strips that are used for different species. For example, a single meter may be used to analyze a first test strip for analyzing samples from a first species, a second test strip for analyzing samples from a second species, and a third test strip for analyzing samples from third species. The type of test strip may be identified by the meter using a calibration pattern disposed on the test strip, as explained above.

After receipt of the sample in the sensor, the analyte in the sample is, e.g., electrooxidized or electroreduced, at the working electrode and the level of signal generated is correlated as analyte concentration. The sensor may be operated with or without applying a potential to the electrodes. In one embodiment, the electrochemical reaction occurs spontaneously and a potential need not be applied between the working electrode and the counter electrode. In another embodiment, a potential is applied between the working electrode and the counter electrode.

### Manufacturing of Analyte Sensor

Analyte sensor or sensor strips discussed above, are sandwiched or layered constructions having first and second substrates spaced apart by a spacer layer and optionally including a mesh layer in the sample chamber defined by the first and second substrates and the spacer layer. Such a construction can be made by combining the various layers together in any suitable manner. An alternate method for making sensor strips as described herein is to mold the sensors.

In general, the method of manufacturing sensor strips involves positioning a working electrode and a reference and/or a counter electrode on the first or the second substrates, contacting at least a portion of the working electrode and/or reference and/or counter electrode with a sensing layer composition.

Optionally, providing a mesh in the sample chamber defined by the first and second substrates and the spacer layer.

Other embodiments and modifications within the scope of the present disclosure will be apparent to those skilled in the relevant art. Various modifications, processes, as well as numerous structures to which the embodiments of the invention may be applicable will be readily apparent to those of skill in the art to which the invention is directed upon review of the specification. Various aspects and features of the invention may have been explained or described in relation to understandings, beliefs, theories, underlying assumptions, and/or working or prophetic examples, although it will be understood that the invention is not bound to any particular understanding, belief, theory, underlying assumption, and/or working or prophetic example. Although various aspects and features of the invention may have been described largely with respect to applications, or more specifically, medical applications, involving diabetic humans, it will be understood that such aspects and features also relate to any of a variety of applications involving non-diabetic humans and any and all other animals. Further, although various aspects and features of the invention may have been described largely with respect to applications involving in vitro disposable single use sensor strips, it will be understood that such aspects and features also relate to any of a variety of sensors that are suitable for use in connection with the body of an animal or a human, such as those suitable for use as partially implanted sensors, such as transcutaneous or subcutaneous sensors or fully implanted in the body of an animal or a human. Finally, although the various aspects and features of the invention have been described with respect to various embodiments and specific examples herein, all of which may be made or carried out conventionally, it will be understood that the invention is entitled to protection within the full scope of the appended claims.

1. An analyte sensor comprising:
a first substrate comprising a first major surface and a second major surface and a second substrate comprising a first major surface and a second major surface such that the second major surface of the first substrate faces the first major surface of the second substrate to provide a sample chamber,
the sample chamber comprising a first electrode disposed on the second major surface of the first substrate or the first major surface of the second substrate and a second electrode disposed on the second major surface of the first substrate or the first major surface of the second substrate;
wherein the sample chamber is positioned at a proximal end of the analyte sensor and wherein a distal end of the analyte sensor is connectable to a meter;
a conductive calibration pattern on the first major surface of the first substrate and/or second major surface of second substrate at or near the distal end, wherein the calibration pattern provides a species-specific calibration information to the meter;
wherein the conductive calibration pattern is one of a plurality of patterns, wherein the plurality of patterns comprise different patterns which provide different species-specific calibration information to a meter.

2. The analyte sensor of claim 1, wherein the sensor further comprises a graphic disposed on the first major surface of the first substrate and/or second major surface of second substrate, wherein the graphic indicates the species compatible for the analyte sensor.

3. The analyte sensor of claim 1, wherein the species-specific calibration information is for a non-human species.

4. The analyte sensor of claim 1, wherein the species-specific calibration information is for a feline, canine, equine, or murine species.

5. The analyte sensor of claim 1, wherein shape of the calibration pattern provides the species-specific calibration information.

6. The analyte sensor of claim 1, wherein resistance of the calibration pattern provides the species-specific calibration information.

7. The analyte sensor of claim 1, wherein the calibration information comprises prompting the meter to require input of a calibration code.

8. A system for analysis of an analyte in a sample, the system comprising:
an analyte sensor comprising:
a first substrate comprising a first major surface and a second major surface and a second substrate comprising a first major surface and a second major surface such that the second major surface of the first substrate faces the first major surface of the second substrate to provide a sample chamber,
the sample chamber comprising a first electrode disposed on the second major surface of the first substrate or the first major surface of the second substrate and a second electrode disposed on the second major surface of the first substrate or the first major surface of the second substrate;
wherein the sample chamber is positioned at a proximal end of the analyte sensor and wherein a distal end of the analyte sensor is connectable to a meter;
a conductive calibration pattern on the first major surface of the first substrate and/or second major surface of second substrate at or near the distal end, wherein the calibration pattern provides a species-specific calibration information to the meter;
wherein the conductive calibration pattern is one of a plurality of patterns, wherein the plurality of patterns comprise different patterns which provide different calibration information to a meter;
a meter comprising a port comprising contacts for contacting the calibration pattern.

9. The system of claim 8, wherein the analyte sensor further comprises a graphic disposed on the first major surface of the first substrate and/or second major surface of second substrate, wherein the graphic indicates the species compatible for the analyte sensor.

10. The system of claim 8, wherein the species-specific calibration information is for a non-human species.

11. The system of claim 8, wherein the species-specific calibration information is for a feline, canine, equine, or murine species.

12. The system of claim 8, wherein shape of the calibration pattern provides the species-specific calibration information.

13. The system of claim 8, wherein resistance of the calibration pattern provides the species-specific calibration information.

14. A method of providing species-specific calibration code on an analyte sensor, the method comprising:
performing sensor calibration on at least one of a batch of analyte sensors to determine a species-specific calibration code for the analyte sensor;
disposing a conductive calibration pattern on a surface of analyte sensors in the batch, wherein the calibration pattern provides the species-specific calibration code to a meter,
wherein the conductive calibration pattern is one of a plurality of calibration patterns, wherein the plurality of patterns comprise different patterns which provide different calibration information to a meter.
